(19) United States
(12) Patent Application Publication
(10) Pub. No.: US 2017/0029568 A1
(43) Pub. Date: Feb. 2, 2017

(54) METHODS OF MANUFACTURE OF SALTS OF HYDROXY-SUBSTITUTED AROMATIC COMPOUNDS AND POLYETHERIMIDES

(71) Applicant: SABIC GLOBAL TECHNOLOGIES B.V., BERGEN OP ZOOM (NL)

(72) Inventor: MANI RAJ CHAULAGAIN, EVANSVILLE, IN (US)

(21) Appl. No.: 15/304,087

(22) PCT Filed: Apr. 15, 2015
(86) PCT No.: PCT/US2015/025946

§ 371 Date: Oct. 14, 2016

Related U.S. Application Data
(60) Provisional application No. 61/979,742, filed on Apr. 15, 2014.

(51) Int. Cl. C08G 73/10; C07C 37/66

(52) U.S. Cl. CPC C08G 73/1071; C07C 37/66; C08G 73/1003

(57) ABSTRACT

A method for the manufacture of a metal salt of a hydroxy-substituted aromatic compound comprises reacting a hydroxy-substituted aromatic compound with a base comprising a metal cation in an aqueous medium to provide a mixture comprising water and a metal salt of the hydroxy-substituted aromatic compound; contacting the mixture with a substantially water-immiscible solvent at a temperature greater than the boiling point of water at a prevailing pressure; introducing an optionally substituted C1-6 aliphatic alcohol; and removing water and the alcohol to provide a slurry comprising the metal salt of the hydroxy-substituted aromatic compound and the water-immiscible solvent.

### BACKGROUND

This disclosure is directed to a method for the manufacture of salts of hydroxy-substituted aromatic compounds, particularly to the manufacture of alkali metal salts of hydroxy-substituted aromatic compounds. The disclosure is also directed to the manufacture of polyetherimides from the prepared salts of hydroxy-substituted aromatic compounds.

Salts of hydroxy-substituted aromatic compounds find varied uses in the industry. For example, bisphenol dialkali salts can be used for the synthesis of polyetherimides via displacement polymerizations.

The existing methods to prepare bisphenol dialkali salts include reacting a bisphenol such as bisphenol A and an alkali hydroxide in an aqueous solution, then adding the aqueous solution containing the formed salt to heated ortho-dichlorobenzene to dry the salt by azeotropic distillation.

However, the synthesis of the dipotassium salt of bisphenol A by this method generates the salt as a hard solid that crystallizes on the walls of the drying vessel. The salt is difficult to remove and it is also impractical to use directly for subsequent reactions. Accordingly, there exists a need to provide alternative methods for preparing salts of hydroxy-substituted aromatic compounds. It would be an advantage if such a method does not result in substantial accumulation of solid salts on the vessel walls.

### SUMMARY

Disclosed is a method for the manufacture of a metal salt of a hydroxy-substituted aromatic compound, the method comprising reacting a hydroxy-substituted aromatic compound with a base comprising a metal cation in an aqueous medium to provide a mixture comprising water and a metal salt of the hydroxy-substituted aromatic compound; contacting the mixture with a substantially water-immiscible solvent at a temperature greater than the boiling point of water at a prevailing pressure; introducing an optionally substituted C₁₋₆ aliphatic alcohol; and removing water and the alcohol to provide a slurry comprising the metal salt of the hydroxy-substituted aromatic compound and the water-immiscible solvent.

In another embodiment, a method for the manufacture of a metal salt of a hydroxy-substituted aromatic compound comprises reacting a hydroxy-substituted aromatic compound with a base comprising a metal cation in an aqueous medium to provide a mixture comprising water and a metal salt of the hydroxy-substituted aromatic compound; contacting the mixture with a substantially water-immiscible solvent at a temperature greater than the boiling point of water at a prevailing pressure; partially removing water and the water-immiscible solvent from the contacted mixture to provide a water-immiscible, solvent-rich phase comprising the metal salt of the hydroxy-substituted aromatic compound and the water-immiscible solvent; introducing an optionally substituted C₁₋₆ aliphatic alcohol to the water-immiscible phase to provide a solution; and separating the water and the isopropanol from the solution to provide a slurry comprising the metal salt of the hydroxy-substituted aromatic compound and the water-immiscible solvent.

In yet another embodiment, a method for the manufacture of a metal salt of a hydroxy-substituted aromatic compound comprises: reacting a hydroxy-substituted aromatic compound with a base comprising a metal cation in an organic medium comprising isopropanol, to provide a mixture comprising a metal salt of the hydroxy-substituted aromatic compound, an optionally substituted C₁₋₆ alcohol, and water produced from the reaction between the hydroxy-substituted aromatic compound and the base; contacting the mixture with a substantially water-immiscible solvent at a temperature greater than the boiling point of water at a prevailing pressure to provide a mixture further comprising the substantially water-immiscible solvent; and removing water and the alcohol from the mixture further comprising the substantially water-immiscible solvent, to provide a slurry comprising the metal salt of the hydroxy-substituted aromatic compound and the water-immiscible solvent.

Also disclosed is a method for the manufacture of a polyetherimide comprising: polymerizing a bis(N-(substituted phthalimido))aromatic compound and an alkali metal salt of a dihydroxy aromatic compound of prepared in accordance with the method described above to form a polyetherimide composition.

The above described and other features are exemplified by the following Drawings, Detailed Description, and Examples.

### BRIEF DESCRIPTION OF THE DRAWINGS

The following is a description of the Figures, which are meant to be exemplary and not limiting.

FIG. 1 is a graph of the MW of polyetherimides as a function of time for displacement polymerization between the K₂BPA powder from Example 3 and a C1PAMI powder from Example 1, wherein the molar ratios of K₂BPA and C1PAMI are 0.95 (0 hr), 0.976 (5 hr) and 0.995 (12 hr) respectively.

FIG. 2 is a graph of Mw of polyetherimides as a function of time for displacement polymerization between the K₂BPA powder from Example 2 and a C1PAMI powder from Example 1 wherein the molar ratios of K₂BPA and C1PAMI are 0.95 (0 hr), 0.976 (5 hr) and 0.995 (7 hr) respectively.

FIG. 3 is a graph of Mw of polyetherimides as a function of time for displacement polymerization between 1:1 molar ratio of K₂BPA slurry from Example 4 and C1PAMI from Example 1;

FIG. 4 is a graph of Mw of polyetherimide in diphenyl sulfone as a function of time for displacement polymerization between 1:1 molar ratio of K₂BPA slurry from Example 5 and C1PAMI from Example 1.

### DETAILED DESCRIPTION

The inventors hereof have discovered that when an optionally substituted C₁₋₆ aliphatic alcohol such as isopropanol is used as a solvent or co-solvent during the manufacture of a metal salt of a hydroxy-substituted aromatic compound, no salts are accumulated on vessel walls during the drying process. The inventors have also found that use of the optionally substituted C₁₋₆ aliphatic alcohol such as isopropanol at the drying stage of the metal salt can effectively dissolve the salts already crystalized on the vessel walls and at the same time prevent the formation of any additional salts on the vessel walls. In an advantageous feature, the methods are effective to produce salts of the hydroxy-substituted aromatic compounds in a fine slurry form, which can be used directly, dried to form a powder, or solvent swapped. In a further advantageous feature, the salts formed by the methods can be used to make polyetherimides via displacement polymerization route without using any phase transfer catalyst.

The metal salts of hydroxy-substituted aromatic compounds are manufactured from the hydroxy-substituted aromatic compound and a base comprising a metal cation. Manufacture can be carried out in an aqueous solvent or organic solvent as further described below. When manufactured in an aqueous solvent, the isopropanol is generally added after salt formation. When manufactured in an organic solvent, the isopropanol can be present during salt formation.

The hydroxy aromatic compound can be a monohydroxy-substituted aromatic compound; a dihydroxy-substituted aromatic compound; a trihydroxy-substituted aromatic compound; a tetrahydroxy-substituted aromatic compound, or a combination comprising at least one of the foregoing. Monohydroxy-substituted aromatic compounds are illustrated by phenol, p-cresol, p-cumylphenol, and the like. Dihydroxy-substituted aromatic compounds are illustrated by dihydroxybenzenes such as hydroquinone, resorcinol, and the like. Dihydroxy-substituted aromatic compounds are further illustrated by bisphenols such as bisphenol A and biphenols such as 4,4′-dihydroxybiphenyl. Trihydroxy-substituted aromatic compounds are illustrated by 1, 3-5-trihydroxybenzene; 1,1,1-tris(4-hydroxyphenyl)ethane (THPE); and the like. Tetrahydroxy-substituted aromatic compounds are illustrated by 2,2-bis(3,4-dihydroxyphenyl)propane; 3,4,3′,4′-tetrahydroxybiphenyl; and the like.

In an embodiment, the dihydroxy-substituted aromatic compound can be an aromatic C₆₋₂₄ monocyclic or polycyclic dihydroxy aromatic compound optionally substituted with 1 to 6 C₁₋₈ alkyl groups, 1 to 8 halogen atoms, or a combination thereof, for example a dihydroxy aromatic compound of formula (1):

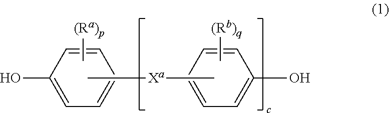

wherein R^{a} and R^{b} are each independently a halogen atom or a monovalent hydrocarbon group and can be the same or different; p and q are each independently integers of 0 to 4; c is 0 to 4, specifically zero or 1; and X^{a} is a bridging group connecting the two aromatic groups, where the bridging group and point of attachment of each C₆ arylene group are disposed ortho, meta, or para (specifically para) to each other on the C₆ arylene group. The bridging group X^{a} can be a single bond, —O—, —S—, —S(O)—, —S(O)₂—, —C(O)—, or a C₁₋₁₈organic bridging group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. A specific example of a dihydroxy aromatic compound is of formula (11a)

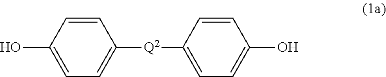

wherein Q² is a single bond, —O—, —S—, —C(O)—, —SO₂—, —SO—, —C_{y}H_{2y}— and a halogenated derivative thereof wherein y is an integer from 1 to 5, including perfluoroalkylene groups. In a specific embodiment Q² is 2,2-isopropylidene and the dihydroxy-substituted aromatic compound is bisphenol A. In another specific embodiment Q² is a single bond and the dihydroxy-substituted aromatic compound is 4,4′-dihydroxybiphenyl.

The base comprising a metal cation can be, for example, an alkali metal hydroxide, an alkaline-earth metal hydroxide (alkali hydroxide), alkali metal carbonate and alkali-earth metal carbonate (alkali carbonate), alkali metal bicarbonate and alkali earth-metal bicarbonate (alkali bicarbonate), or a combination comprising at least one of the foregoing. In an embodiment an alkali metal hydroxide is used as the base comprising a metal cation. In yet another embodiment the base is sodium hydroxide or potassium hydroxide.

The base can be used in any convenient form. When the reaction between the hydroxy-substituted aromatic compound and the base is conducted in an aqueous medium, the base is typically used as an aqueous solution. In an illustrative example an aqueous solution containing 30-70% by weight of the base in water is suitable. Solutions comprising 50% by weight concentration of the base are readily available and their use can be preferred. When the reaction between the hydroxy-substituted aromatic compound and the base is conducted in an organic medium comprising isopropanol, a solid base is used. Illustrative, non-limiting examples of solid bases comprise solid alkali metal hydroxides such as sodium hydroxide and potassium hydroxide.

The aqueous medium as described herein refers to a medium comprising at least 10 weight percent (wt. %) of water based on the total weight of the aqueous medium. Further the aqueous medium in the present context refers to a medium in which a hydroxy-substituted aromatic compound reacts to form a metal salt in the presence of a base comprising a metal cation. In an embodiment the aqueous medium is such that the hydroxy-substituted aromatic compound is at least partially soluble. In another embodiment the aqueous medium is such that a hydroxy-substituted aromatic compound is essentially completely soluble in the aqueous medium. In another embodiment the hydroxy-substituted aromatic compound is at least partially insoluble in the aqueous medium and is solubilized in the presence of a base comprising a metal cation, on the formation of the corresponding metal salt of the hydroxy-substituted aromatic compound.

In an embodiment the aqueous medium comprises water and, optionally, at least one substantially water-miscible organic solvent (i.e., a co-solvent). Substantially water-miscible in the present context refers to a solubility of the organic co-solvent in water of 90 wt. % or greater, or 95 wt. % or greater, or 98 wt. % or greater, or 99 wt. % or greater under the reaction conditions. Water-miscible organic solvents are well-known in the art and typically include hydroxy-substituted aliphatic compounds such as methanol, ethanol, propanol, isopropanol, butanol, ethylene glycol, propylene glycol, and combinations comprising at least one of the foregoing water-miscible organic solvents. In an embodiment when the solvent medium comprises water and at least one substantially water-miscible organic solvent, then the amount of the water-miscible organic solvent can be 10 to 90 wt. %, or 60 to 90 wt. %, or 80 to 95 wt. %, based on the total weight of water and the water-miscible organic solvent. In some embodiments the amount of the water-miscible organic solvent is sufficient to essentially effect complete solubility of hydroxy-substituted aromatic compound in a mixture with water.

The organic medium as described herein refers to a medium comprising at least one of an optionally substituted C₁₋₆ aliphatic alcohol. Suitable alcohols include methanol, ethanol, propanol, isopropanol, butanols, pentanols, and hexanols. Optionally the alcohol is substituted with a halogen. Further the organic medium comprises less than 5 wt. %, less than 2 wt. %, or less than 1 wt. % of water based on the total weight of the organic medium. The reaction between the hydroxy-substituted aromatic compound and the base generates water. No other water is added to the organic medium.

In the method, the hydroxy-substituted aromatic compound is reacted with the base comprising a metal cation in the aqueous or organic medium. The reaction can be performed using stoichiometric amounts, wherein the base and the hydroxy-substituted aromatic compound are present in amounts corresponding to a molar ratio of base to hydroxy-substituted aromatic compound which in an embodiment deviates from ideal stoichiometry by no more than 5.0 mole %, no more than 3 mole %, or no more than 1.0 mole %. In preferred embodiment, the stoichiometry varies by no more than 0.4 mole %. In another embodiment the molar ratio deviates from ideal stoichiometry by no more than 0.2 mole %.

The reaction of the hydroxy-substituted aromatic compound and the base can be performed in the aqueous medium or the organic medium at a temperature which provides for the efficient conversion of the hydroxy-substituted aromatic compound to the corresponding metal salt. In an embodiment the temperature is 50° C. to 150° C., or 70° C. to 100° C., or 80° C. to 100° C.

The reaction of the hydroxy-substituted aromatic compound with the base can be performed in the aqueous medium or the organic medium for a period of time sufficient to obtain the desired degree of conversion to the metal salt. In various embodiments the contact time depends upon a number of factors such as the amounts of hydroxy-substituted aromatic compound and the base used. In a particular embodiment the contact time is for greater than 1 hour, or for 1.5 hours to 3 hours. Appropriate contact times depend upon reaction temperatures and the nature of the reactants, and this can be determined by one skilled in the art, without undue experimentation.

The reaction of the hydroxy-substituted aromatic compound with the base in an aqueous medium or in an organic medium can be performed under an inert atmosphere, such as under nitrogen, argon, or helium.

The reaction of the hydroxy-substituted aromatic compound with the base in an aqueous medium or in an organic medium can be performed at a solids level of greater than 5%, wherein the solids level is the weight of salt of the hydroxy-substituted aromatic compound divided by the sum of weight of the reactants and weight of the aqueous or organic medium. In another embodiment the solids level is greater than 15%, or greater than 25%. The course of the reaction can be monitored by known methods.

Once the salt is formed, the mixture comprising the metal salt in the aqueous or organic medium is maintained at a temperature effective to maintain the metal salt of hydroxy-substituted aromatic compound in solution. The metal salt solution is then contacted with a substantially water-immiscible solvent. “Substantially water-immiscible solvent” as used herein means that the solvent is soluble to the extent of less than 10% by weight, or less than 5% by weight, or less than 1% by weight in water; or that water is soluble to the extent of less than 10% by weight or less than 5% by weight or less than 1% by weight in the solvent. The water-immiscible solvent can be contained in a drying vessel. Before and during contacting with the hydroxy-substituted aromatic metal salt solution, the substantially water-immiscible solvent is maintained at a temperature that is greater than the boiling point of the aqueous medium or organic medium under the prevailing pressure, and preferably greater than the boiling point of water under the prevailing pressure. The temperature at which the water-immiscible solvent is maintained before and during addition can be 75 to 220° C., or 100 to 200° C., or 140° C. to 175° C.

The water-immiscible solvents can be compounds having the formula

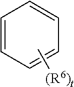

wherein each R⁶ is independently halogen, C₁₋₆ aliphatic radical, or C₃₋₁₂ aromatic radical; and t is an integer from 1 to 6. Suitable water-immiscible solvents include toluene, xylene, benzene, phenetole, anisole, veratrole, diphenylsulfone, chlorobenzene, bromobenzene, ortho-dichlorobenzene, meta-dichlorobenzene, para-dichlorobenzene, 1,3,5-trichlorobenzene, and 1,2,4-trichlorobenzene. In an embodiment, the water-immiscible solvents include ortho-dichlorobenzene or xylenes. Combinations of the water-immiscible solvents can be used. In various embodiments suitable water-immiscible solvents are those having a boiling point at atmospheric pressure of greater than 790° C., or greater than 150° C., or greater than 170° C. In some embodiments suitable solvents also have a specific gravity of 0.75 to 1.5. In some particular embodiments suitable water-immiscible solvents have a specific gravity of greater than 1.25.

The solution comprising the metal salt of the hydroxy-substituted aromatic compound in the aqueous medium can be contacted with the water-immiscible solvent in various ways. In various embodiments the metal salt in the aqueous medium or the organic medium can be either fed in drop-wise into the water-immiscible solvent or it can be sprayed into the water-immiscible solvent.

The contacting of the metal salt of the hydroxy-substituted aromatic compound in an aqueous medium or an organic medium with a water-immiscible solvent in the drying vessel, can be carried out under agitation. The agitation can be maintained either for the entire time period required for drying or for a portion of the entire time period required for drying. In some particular embodiments the vessel comprises a stirred tank with at least one stirring shaft agitator. The degree of agitation is typically such as not to favor formation of salt cake in or on any part of the vessel or agitator which can be difficult to remove. In various embodiments the vessel comprises baffles beneath the surface of the water-immiscible solvent. At least two baffles can be present. In an embodiment greater than two baffles are present and in other embodiments between two and four baffles can be present. The design of the baffles is such that build-up of salt is not facilitated. In some embodiments embodiment the baffles are substantially vertical and are attached to the sides of the vessel, optionally starting at the tangent line from a curved surface at the bottom of the vessel should the vessel possess a curved bottom. Any baffle is attached to the side of the vessel at only one, two, or three or more spots on the baffle so that there is at least a partial gap between any baffle and the side of the vessel such that salt can pass through the gap and not collect to a significant extent against any baffle.

The drying vessel containing water-immiscible solvent can be fitted with equipment comprising at least one pipe and at least one spray nozzle for introduction of the aqueous medium or organic medium comprising the metal salt of the hydroxy-substituted aromatic compound into the vessel. In an embodiment at least one pipe fitted with at least one spray nozzle conveys the aqueous medium or organic medium comprising the metal salt of the hydroxy-substituted aromatic compound from the vessel in which the metal salt was prepared into the drying vessel containing water-immiscible solvent. One, two, three, four, or more spray nozzles can be used for introduction of the aqueous medium or organic medium comprising the metal salt of the hydroxy-substituted aromatic compound into the drying vessel. In some embodiments one to ten or two to four spray nozzles for introduction of aqueous medium comprising metal salt of hydroxy-substituted aromatic compound are used. In an embodiment the spray nozzle or nozzles can project into the drying vessel from the top of the drying vessel. In another embodiment the spray nozzle or nozzles can be mounted flush with the top of the drying vessel. The spray of the aqueous medium or the organic medium comprising the metal salt of the hydroxy-substituted aromatic compound is directed to the surface of the water-immiscible solvent within the vessel, and preferably away from any agitator shaft and the sides of the vessel. The distance between any spray nozzle and the surface of the water-miscible solvent level can be any convenient distance to provide for spraying of the aqueous medium comprising the metal salt of the hydroxy-substituted aromatic compound into the vessel and formation of the vapor stream described above, with efficient use of the vessel space. In some embodiments a spray nozzle is at a distance of 0.15 to 3.0 meters or 0.3 to 2.5 meters or 0.3 to 1.5 meters above the surface of the water-immiscible solvent.

Any dead space cavities in the vessel can be heated externally or flushed with dry/hot solvent to prevent any accumulation of water or metal salt cake therein. In an embodiment the vessel sides and top are traced with heating elements to provide external heating. In other embodiments provision can be made for contacting the top of the vessel and any dead spaces with hot water-immiscible solvent by spraying water-immiscible solvent therein. The water-immiscible solvent can comprise fresh solvent or solvent returned from condensate which was originally distilled from the vessel along with aqueous medium, or both fresh and returned solvent. The spraying of water-immiscible solvent can be performed with equipment comprising at least one pipe and at least one spray nozzle for introduction of water-immiscible solvent. One, two, three, four, or more spray nozzles can be used for introduction of water-immiscible solvent into the vessel. In some embodiments one to ten or two to 4 spray nozzles for introduction of water-immiscible solvent are used. In an embodiment the spray nozzle or nozzles for introduction of water-immiscible solvent can project into the drying vessel from the top of the vessel. In another embodiment the spray nozzle or nozzles for introduction of water-immiscible solvent can be mounted flush with the top of the vessel to help prevent caking of salt. Water-immiscible solvent can be sprayed into the vessel as desired and in an embodiment is sprayed into the vessel simultaneously with spraying of aqueous medium or organic medium comprising metal salt of hydroxy-substituted aromatic compound through separate spray nozzles.

The rate of introduction of the aqueous medium or organic medium comprising the metal salt of a hydroxy-substituted aromatic compound into the vessel containing the water-immiscible solvent depends upon a number of factors, including, but not limited to, vessel size, temperature of the water-immiscible solvent, and amount of heating capability, and can be determined by one skilled in the art without undue experimentation. If the rate of introduction is too high, then the temperature of the water-immiscible solvent can fall and the metal salt of hydroxy-substituted aromatic compound can tend to cake. On the other hand, if the rate of introduction is too low, then process economics can be less favorable. In general, the rate of introduction of aqueous medium or the organic medium comprising metal salt of hydroxy-substituted aromatic compound into the drying vessel containing water-immiscible solvent is as fast as possible to promote rapid formation of vapor stream without excessive caking of the salt. In particular embodiments the aqueous medium or the organic medium comprising the metal salt of a hydroxy-substituted aromatic compound is introduced into the vessel in such a manner that the medium does not impact the walls of the vessel or any stirrer shaft.

Heat can be provided to the water-immiscible solvent using any convenient method. In some embodiments heat is provided to the water-immiscible solvent by circulating the solvent through a heat exchanger, for example a tube-shell heat exchanger. In some other particular embodiments the heat exchanger is a spiral heat exchanger or a self-cleaning reboiler. The rate of flow of the water-immiscible solvent-salt mixture through the heat exchanger is such that turbulent flow is achieved to prevent fouling of the heat exchanger by solid salt. The rate of flow depends upon a number of factors, including, but not limited to, the concentration of salt therein and the temperature, and can be determined without undue experimentation by one skilled in the art.

In an embodiment the drying vessel containing the water-immiscible solvent into which the aqueous medium or the organic medium comprising the metal salt of a hydroxy-substituted aromatic compound is introduced can be under a positive pressure so that the temperature of water-immiscible solvent can be maintained above its normal boiling point at atmospheric pressure. For example, the drying vessel can be maintained at a pressure of 0 to 100 psig, or 0 to 50 psig, or 0 psig to 25 psig, wherein 0 psig refers to atmospheric pressure. In another embodiment the vessel holding the water-immiscible solvent into which the aqueous medium or organic medium comprising metal salt of hydroxy-substituted aromatic compound is introduced can be maintained at sub-atmospheric pressure. Operating under sub-atmospheric pressure tends to lower the distillation temperature of the mixture for formation of vapor stream, and can help limit decomposition of the metal salt of hydroxy-substituted aromatic compound, which can occur at least to some extent at elevated temperatures depending upon the identity of the salt.

It is appreciated that once the reaction between the hydroxy-substituted aromatic compound and the base is completed in the aqueous medium, an optionally substituted C₁₋₆ aliphatic alcohol can be introduced. In an embodiment, the aliphatic alcohol is introduced before contacting the product mixture with a substantially water-immiscible solvent. In another embodiment, the aliphatic alcohol is introduced after contacting the product mixture with a substantially water-immiscible solvent.

When the salts of the hydroxy-substituted aromatic compound are formed in an aqueous medium, water and the water-immiscible solvent are partially removed from the drying vessel to provide a water-immiscible, solvent-rich phase. In an embodiment, the water-immiscible phase comprises the metal salt of the hydroxy-substituted aromatic compound, the water-immiscible solvent, and less than 40 wt. %, less than 30 wt. %, less than 20 wt. %, or less than 10 wt. % of water, based on the total weight of the water-immiscible solvent-rich component. An optionally substituted C₁₋₆ aliphatic alcohol such as isopropanol is then introduced into the water-immiscible, solvent-rich phase. The amount of the aliphatic alcohol is selected to provide the advantages described herein, that is, formation of a free flowing slurry. The amount can also be selected to prevent substantial salt deposition on the surfaces of the drying or other vessel. The water and the aliphatic alcohol are then separated from the solution to form a slurry comprising the metal salt of the hydroxy-substituted aromatic compound and the water-immiscible solvent.

When the salts are formed in an organic medium comprising an aliphatic alcohol, the aliphatic alcohol and water produced from the reaction between the hydroxy-substituted aromatic compound and the base can be removed from the drying vessel under reduced pressure to form a slurry comprising the metal salt of the hydroxy-substituted aromatic compound and the water-immiscible solvent.

In an embodiment, the drying vessel is equipped with a vapor handling system comprising a partial reflux condenser. The vapor stream that is formed during the contact of the aqueous medium or organic medium comprising the metal salt of the hydroxy-substituted aromatic compound with the water-immiscible solvent is introduced into the vapor handling system. The partial reflux condenser is typically maintained at a temperature below the boiling point of the water-immiscible solvent under the prevailing conditions and above the boiling point of water under the prevailing conditions, which results in the separation of the vapor stream to provide a water-rich component and a water-immiscible solvent-rich component. The water-immiscible solvent rich component can be condensed in the vapor handling system and returned back into the drying vessel.

In some embodiments, salts accumulate on the walls of the drying vessel during the above process. In order to dissolve the solid salts and/or to prevent the formation of additional solid salts on the vessel walls, isopropanol can be introduced to the water-immiscible phase to form a solution. In an embodiment, the temperature of the water-immiscible phase is lowered to 75 to 120° C. before isopropanol is added. Subsequently, isopropanol and remaining water are removed under reduced pressure to form a slurry comprising the metal salt of the hydroxy-substituted aromatic compound and the water-immiscible solvent.

In an embodiment the slurry comprising the metal salt of a hydroxy-substituted aromatic compound in the water-immiscible solvent, is obtained at a solids level in the water-immiscible solvent of 5 to 35 wt. %, or 10 to 30 wt. %, or 20 to 30 wt. %. The weight percent of solids in the water-immiscible solvent is based on the total weight of the contents left behind in the drying vessel.

Before, during or after transfer to another vessel, or before use in any subsequent process such as in a polymerization reaction, the slurry comprising the metal salt of a hydroxy-substituted aromatic compound can optionally be subjected to at least one drying step to remove any residual water. The drying step includes, but is not limited to, combination with additional water-immiscible solvent and distillation, optionally at reduced pressure, or distillation of water-immiscible solvent from the mixture comprising water-immiscible solvent and metal salt, optionally with concomitant addition of dry water-immiscible solvent at approximately the same rate so as to keep the solvent amount roughly constant. Dry water-immiscible solvent in the context of the present process means solvent with less than 100 parts per million (“ppm”) water. In an embodiment at least one drying step takes place in the drying vessel in which the metal salt of a hydroxy-substituted aromatic compound is prepared. In other embodiments the slurry of the salt of the hydroxy-substituted aromatic compound in the water-immiscible solvent can be transferred from the vessel to at least one other vessel for an additional drying step. In an embodiment the amount of water remaining in the salt-containing water-immiscible solvent after one or more drying steps is less than 100 ppm, preferably less than 60 ppm, more preferably less than 40 ppm with respect to the weight of the dry salt present. The amount of water in the salt-containing water-immiscible solvent can be determined using known methods.

In an embodiment, the salt product is separated from the water-immiscible solvent as a powder. Any known methods can be used. In particular embodiments, separation can be effected by filtration, centrifugation, distillation, or like methods. Remaining traces of water-immiscible solvent in the salt can be removed, if desired, by methods such as vacuum drying, drying under a nitrogen atmosphere or similar operation. It is, however, often convenient to employ the salt in a slurry form in the water-immiscible solvent without isolation of the salt. For example, the salt can be used in slurry form in a subsequent reaction in which the salt is a reactant.

In another embodiment, the water-immiscible solvent in the slurry containing the salt product can be exchanged with a polar aprotic solvent by introducing a polar aprotic solvent to the slurry; and removing the water-immiscible solvent to provide a composition comprising the polar aprotic solvent and the metal salt of the hydroxy-substituted aromatic compound. Specific examples of the polar aprotic solvent include diphenyl sulfone, dimethylformamide (DMF), dimethylacetamide (DMAc), dimethylsulfoxide (DMSO), tetramethylene sulfone (sulfolane), and N-methylpyrrolidinone (NMP). Diphenyl sulfone and tetramethylene sulfone are specifically mentioned.

The process for making metal salt described herein can be performed in batch mode, continuous mode or semi-continuous mode. The metal salt of hydroxy-substituted aromatic compound can be used in one or more subsequent reactions to form materials incorporating structural units derived from the hydroxy-substituted aromatic compound. In a particular embodiment a slurry of the metal salt in water-immiscible solvent can be used in a reaction to form a monomer for use in condensation polymerization. In another embodiment a powder comprising the metal salt in water-immiscible solvent can be used directly as a monomer in condensation polymerization. In yet another embodiment a composition comprising the metal salt and the polar aprotic solvent such as diphenyl sulfone can be used directly as a monomer in condensation polymerization.

The metal salt of hydroxy-substituted aromatic compound in water-immiscible solvent can be used directly as a monomer in the preparation of polyethers such as polyetherimides, polyethersulfones, polyetherimidesulfones, polyetherketones, polyetheretherketones, and the like. In an illustrative example the bis(sodium) salt or bis(potassium) salt of a dihydroxy-substituted aromatic compound such as bisphenol A can be used as a monomer to form a polyetherimide by reaction with at least one bis(N-(substituted phthalimido))aromatic compound. Suitable substituents on the bis(N-(substituted phthalimido))aromatic compounds include any that can be displaced in a polymerization reaction with the metal salt of a hydroxy-substituted aromatic compound. In particular embodiments suitable substituents are nitro, halogen, chloro, and bromo. Advantageously, in an embodiment, the polymerization reaction can be carried out without using any phase transfer catalyst. The polymerization reaction can be performed in at least one solvent of low polarity. In various embodiments the solvent has a boiling point above 150° C. in order to facilitate the displacement reaction, which typically requires of 125 to 250° C. Solvents of this type include ortho-dichlorobenzene, para-dichlorobenzene, dichlorotoluene, 1,2,4-trichlorobenzene, diphenyl sulfone, phenetole, anisole, veratrole and combinations comprising at least one of the foregoing. Often the polymerization reaction is performed under conditions such that less than 50 parts per million water is present with respect to dry weight of hydroxy-substituted aromatic compound salt.

It is appreciated that in an embodiment, tetramethylene sulfone (sulfolane) can be used as an alternative to diphenyl sulfone. Accordingly, whenever diphenyl sulfone is mentioned, it can be replaced with tetramethylene sulfone (sulfolane).

The methods of the manufacture of salts of hydroxy-substituted compounds and the methods of the manufacture of polyetherimide from the produced salts are further illustrated by the following non-limiting examples.

### EXAMPLES

### Materials

The materials in Table 1 were used or made in the following Examples.

TABLE 1

| Acronym | Description | Source |
|---|---|---|
| BPA | 2,2-Bis(4-hydroxyphenyl)propane, (Bisphenol A) | Hexion |
| K₂BPA | Bisphenol, dipotassium salt | Examples |
| KOH | Potassium hydroxide | Acculute |
| IPA | Isopropyl alcohol (isopropanol) | Aldrich |
| DPS | Diphenyl sulfone | |
| o-DCB | ortho-Dichlorobenzene | Fischer |
| ClPA | Mixture of 3-chlorophthalic anhydride and | SABIC |
| | 4-chlorophthalic anhydride | |
| mPD | meta-Phenylene diamine | DuPont |
| AcOH | Acetic acid | Aldrich |
| ClPAMI | 1,3-bis[N-(4-chlorophthalimido)]benzene | Examples |
| PEI | Polyetherimide | Examples |
| H₃PO₄ | Phosphoric acid | Fischer |

### Property Testing

Gel Permeation Chromatograph (GPC) analysis was carried out as follows. In a 20 ml glass vial, about 20 mg of the polymer sample was taken and dissolved into a quench solution (3.5 L CH₂Cl₂+120 mL AcOH+30 mL o-DCB) followed by filtration with 0.25 micron filter into an HPLC vial. The solution was analyzed by GPC with polystyrene standards (HPLC 2695, Waters GPC software using 2487 Dual absorbance detector of wavelength 254 nm and Mixed Bed C, PLgel 5 micrometer, 300×7.5 mm, P/N 1110-6500 column).

### Example 1

### Synthesis of C1PAMI in O-DCB and Isolation

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap. Chlorophthalic anhydride (C1PA) (35.028 g, 0.1919 moles, 2.008 equiv) and meta-phenylene diamine (mPD) (10.333 g, 0.0955 moles, 1.0 equiv, APHA=35) were charged under nitrogen. o-DCB (288 mL) was degassed at 130° C. in a separate 3-neck flask for at least 30 min. The degassed o-DCB was cannulated into the flask (to make a 10% solid mixture). The reaction flask was then immersed into the oil bath and heated to 145° C. The reaction generated a gel when the temperature reached 125° C. Slow and continuous heating/stirring (100-150 rpm) broke the gel into a slurry. The temperature of the oil bath was increased to 185° C.; and the reaction mixture was stirred for a total of 6 hr. Stripping off 77 mL o-DCB (and water) provided a C1PAMI slurry in o-DCB with a 13% solids content. The slurry was free from residual monoamines and C1PAs. Karl-Fisher analysis was used to test the moisture content (<80 ppm). The C1PAMI slurry in o-DCB was filtered through a 2.7 micron filter paper in a Buchner Funnel. The solid cake was then dried in a vacuum oven at 160° C. for 14 hr. The dry solid was crushed into powder.

### Example 2

### Synthesis of K₂BPA Powder Using Aqueous KOH, BPA, Xylenes and IPA and Polymerization with C1PAMI in DPS

This example demonstrates synthesis of the powdered K₂BPA salt with help of IPA and using Xylenes as non-polar solvent and polymerization with C1PAMI made in Example 1 in DPS as solvent to make polyetherimide (PEI) without using phase transfer catalyst (PTC). The example also shows the control of Mw of the PEI polymer by varying the molar ratio of the K₂BPA salt/C1PAMI molar ratio.

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then charged with 11.4145 g BPA (0.05 moles, 1 equiv.) and 0.1 moles aqueous KOH solution. The overhead stirrer was turned on and the flask was immersed into the oil bath at 80° C. The stirring was continued for 1 hr. Another 500 mL 3-neck flask with the above set-up was charged with 200 mL xylenes and heated to 140° C. The aqueous salt solution was slowly cannulated into the flask containing the heated xylenes and xylenes/water was stripped off into the Dean Stark trap. After removing majority of the water, the salt precipitated out as solid on the wall of the flask. The temperature of the flask was decreased to 100° C. and 100 mL isopropanol was added to the flask. The solid dissolved again forming a solution. Upon stripping of the solvents while slowly increasing the temperature to 150° C., the solution started to become cloudy. After IPA and the remaining water were removed, a K₂BPA salt slurry in xylenes was formed. The salt was converted into a dry powder by further stripping off xylenes. The power was dried in a vacuum oven at 140° C. for 12 hr.

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then immersed into an oil bath at 170° C. and DPS (80 g) was added. Once the DPS was completely molten, stirrer was turned on and 8.658 g of the dry powdered C1PAMI of Example 1 (0.0198 moles, 1.0 equiv) was added forming a slurry of C1PAMI in DPS. To this slurry, K₂BPA salt powder made in this example (5.984 g, 95.7% solid, 0.0196 moles, 0.95 equiv.) prepared in Example 1 was added. The temperature of the reaction mixture was increased from 170° C. to 220° C. The mixture first became thick solid and then became thinner. Mw build was monitored by GPC analysis and the results are shown in FIG. 2. Additional K₂BPA salt was added at 5 hr and 12 hr to adjust the molar ratio of K₂BPA to C1PAMI to 0.976 (5 hr) and 0.995 (12 hr) respectively to reach the final Mw, with PDI 2.67.

The reaction was quenched with phosphoric acid (85%, 670 mg) at 170° C. and stirred for 30 min. The mixture was then transferred into a 500 mL glass jar with a Teflon cap and cooled. Methylene chloride (200 mL) was added into the solidified polymer solution. The mixture was shaken to convert the solid into a suspension. The suspension was filtered through 0.7 micron glass fiber filter paper in a Buchner Funnel to remove the precipitated solid. The clear polymer solution in DPS and methylene chloride was slowly added to 500 mL acetone with constant agitation by a homogenizer to precipitate the polyetherimide which was filtered and washed with 500 mL acetone twice to provide a polyetherimide powder, which was subsequently dried in vacuum at room temperature.

### Example 3

### Synthesis of K₂BPA Powder Using BPA, aq KOH, o-DCB and IPA and Polymerization with C1PAMI in DPS

This example demonstrates synthesis of the powdered K₂BPA salt with help of IPA and using ODCB as non-polar solvent and polymerization with C1PAMI made in Example 1 in DPS as solvent to make polyetherimide without using phase transfer catalyst (PTC). The example also shows the control of Mw of the polyetherimide polymer by varying the molar ratio of the K₂BPA salt/C1PAMI molar ratio.

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then charged with 11.4145 g BPA (0.05 moles, 1 equiv.) and 0.1 moles aqueous KOH solution. The overhead stirrer was turned on and the flask was immersed into the oil bath at 80° C. The stirring was continued for 1 h. Another 500 mL 3-neck flask with the above set-up was charged with 200 mL o-DCB and heated to 160° C. The aqueous salt solution was slowly cannulated into the flask containing heated o-DCB. After stripping off majority of the water, the salt started to precipitate on the wall of the flask. The temperature of the flask was then decreased to 100° C. and 100 mL isopropyl alcohol (IPA) was added slowly while stirring. The solid dissolved again. Upon stripping of the solvents while slowly increasing the temperature to 160° C., the solution started to become cloudy. After IPA and remaining water were removed, a K₂BPA salt free-flowing slurry in o-DCB was obtained wherein the salt did not adhere to the sides of the vessel. It should be appreciated that salt sticking to the sides of the vessel impedes subsequent operations that uses the salt slurry, and that it is difficult to remove trace water, which is necessary for the successful use of the salt in any subsequent polymerization reaction. The salt was converted into a dry powder by further stripping off o-DCB. The power was dried in a vacuum oven at 150° C. for 12 hr.

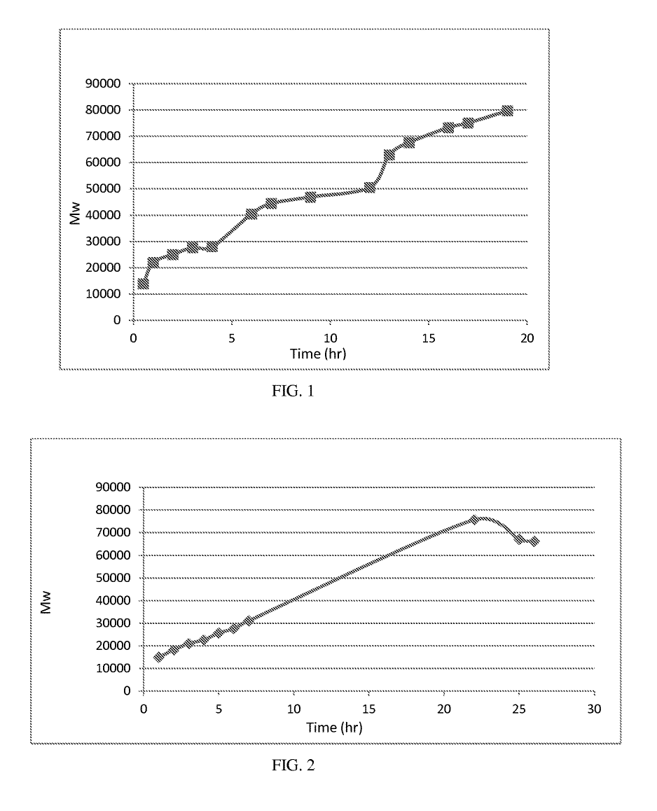

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then immersed into an oil bath at 170° C. and DPS (80 g) was added. Once the DPS was completely molten, stirrer was turned on and 8.658 g of the dry powdered C1PAMI from Example 1 (0.0198 moles, 1.0 equiv) was added forming a slurry of C1PAMI in DPS. To this slurry, K₂BPA salt powder (5.984 g, 95.7% solid, 0.0196 moles, 0.95 equiv.) prepared in Example 2 was added. The temperature of the reaction mixture was increased from 170° C. to 220° C. The mixture first became thick solid and then became thinner. Mw build was monitored by GPC analysis and the results are shown in FIG. 1. Additional K₂BPA salt was added at 5 hr and 12 hr to adjust the molar ratio of K₂BPA to C1PAMI to 0.976 (5 hr) and 0.995 (12 hr) respectively to reach the final Mw 79650 with PDI 3.05. The polymer was isolated as described in example 2.

### Example 4

### Synthesis of K2BPA Slurry in DPS Using BPA, aq KOH, o-DCB, IPA and DPS and Polymerization with C1PAMI

This example demonstrates synthesis of a slurry of K₂BPA salt in DPS with help of IPA and using o-DCB as non-polar solvent and polymerization with C1PAMI made in Example 1 in DPS as solvent to make polyetherimide without using phase transfer catalyst (PTC).

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then charged with 11.4145 g BPA (0.05 moles, 1 equiv.) and 0.1 moles aqueous KOH solution. The overhead stirrer was turned on and the flask was immersed into the oil bath at 80° C. The stirring was continued for 1 hr. Another 500 mL 3-neck flask with the above set-up was charged with 200 mL o-DCB and heated to 160° C. The aqueous salt solution was slowly cannulated into the flask containing heated o-DCB. After stripping off majority of the water, the salt started to crash out as solid on the wall of the flask. The temperature of the flask was then decreased to 100° C. and 100 mL IPA was added slowly while stirring. The solid dissolved again. Upon stripping of the IPA and remaining water while slowly increasing the temperature to 160° C., the solution started to become cloudy and then formed a slurry in o-DCB. Diphenyl sulfone (DPS) (100 g) was added to the slurry at 180° C. and o-DCB was completely stripped off providing a K₂BPA slurry in DPS.

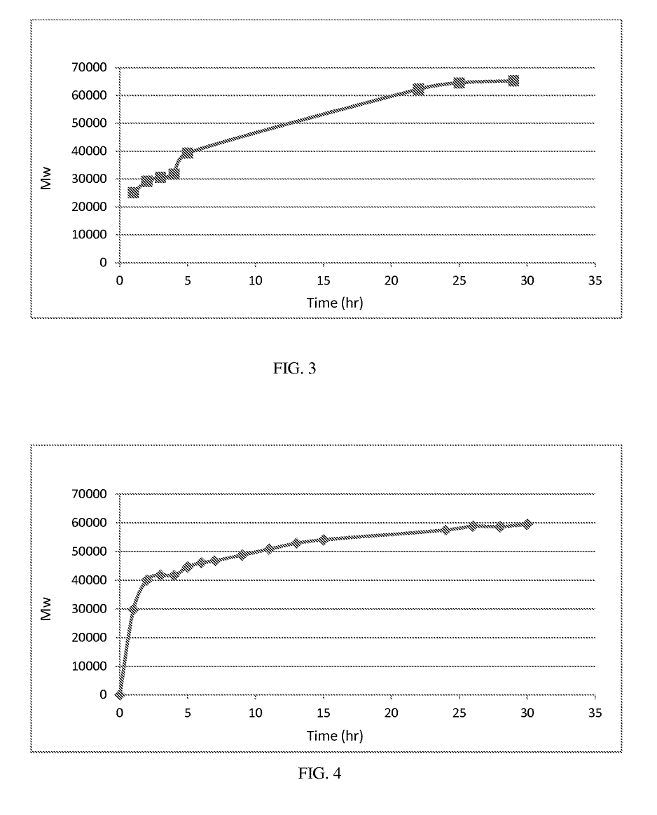

To the salt slurry in DPS at 180° C., C1PAMI powder from Example 1 (21.863 g, 0.5 moles 1 equiv) was added. The temperature of the bath was increased to 200° C. The mixture first became thick solid and then became thinner. Mw build was monitored by GPC analysis with polystyrene standard as shown in FIG. 3. Mw plateau formed at 65270 with PDI 2.68 in 8 hr. The polymer was isolated as described in example 2.

### Example 5

### Synthesis of K2BPA Slurry in DPS Using BPA, KOH Pellets, o-DCB, IPA and DPS and Polymerization with C1PAMI

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then charged with 10.933 g BPA (0.479 moles, 1.0 equiv.) and 6.177 g solid KOH pellets (0.0958 moles, 86% solid, 2.0 equiv.) and 100 mL isopropanol. The overhead stirrer was turned on and the flask was immersed into the oil bath at 80° C. The solid particles slowly dissolved into the solution. The stirring was continued for 1 h. o-DCB (100 mL) was added to the solution and the temperature was slowly increased to 150° C. and o-DCB/water/IPA was stripped off into the Dean-Stark trap. After the isopropanol and water were removed, a K₂BPA salt slurry in o-DCB was formed. The temperature was increased to 170° C. and 80 g diphenyl sulfone added to the slurry while stirring, again without sticking. The o-DCB was stripped off completely providing a K₂BPA salt free flowing slurry in DPS.

To the salt slurry in DPS at 180° C., C1PAMI powder from Example 1 (21.863 g, 0.5 moles 1 equiv) was added. The temperature of the bath was increased to 200° C. The mixture first became thick solid and then became thinner. Mw build was monitored by GPC analysis with polystyrene standard as shown in FIG. 4. The polymer was isolated as described in Example 2. The Mw plateau was formed at 57344 Dalton with PDI 2.61 in 8 hr. The polymer was isolated as described in example 3.

### Example 6

### Synthesis of Na₂BPA Slurry in DPS Using BPA, aq NaOH, Xylenes and DPS and Polymerization with C1PAMI

This example demonstrates synthesis of a slurry of N₂BPA salt in DPS using o-DCB as non-polar solvent and polymerization with C1PAMI made in Example 1 in DPS as solvent to make polyetherimide.

A 500 mL 3-neck round bottomed flask (24/40) was equipped with an overhead stirrer. The flask was also connected to a nitrogen sweep and a nitrogen blanket. The nitrogen blanket was connected to a bubbler via a Dean-Stark trap with its arm wrapped in a heating tape. The flask was then charged with 11.4145 g BPA (0.05 moles, 1 equiv.) and 0.1 moles aqueous NaOH solution. The overhead stirrer was turned on and the flask was immersed into the oil bath at 80° C. The stirring was continued for 1 h. Another 500 mL 3-neck flask with the above set-up was charged with 200 mL xylenes and heated to 140° C. The aqueous salt solution was slowly cannulated into the flask containing heated xylenes stripping off the water into the Dean-Stark trap. After removing majority of the water, the salt solution turned into a slurry. Once the salt is free of water, diphenyl sulfone (DPS) (100 g) was added to the slurry and xylenes was completely stripped off providing a Na₂BPA slurry in DPS.

To the salt slurry DPS at 180° C., C1PAMI powder (21.863 g, 0.5 moles 1 equiv) was added. The temperature of the bath was increased to 200° C. The mixture first became thick solid and then became thinner. Mw build was monitored by GPC analysis with polystyrene standard. Mw formed a plateau at 20 hr of 3 kDalton. Addition of 1 mol % hexaguanidinium Chloride (HEGC1) increased the Mw further to 70725 Dalton with PDI 2.82.

The invention is further illustrated by the following embodiments, which are non-limiting.

Embodiment 1: A method for the manufacture of a metal salt of a hydroxy-substituted aromatic compound, the method comprising: reacting a hydroxy-substituted aromatic compound with a base comprising a metal cation in an aqueous medium to provide a mixture comprising water and a metal salt of the hydroxy-substituted aromatic compound; contacting the mixture with a substantially water-immiscible solvent at a temperature greater than the boiling point of water at a prevailing pressure; introducing an optionally substituted C₁₋₆ aliphatic alcohol; and removing water and the alcohol to provide a slurry comprising the metal salt of the hydroxy-substituted aromatic compound and the water-immiscible solvent.

Embodiment 2: A method for the manufacture of a metal salt of a hydroxy-substituted aromatic compound, the method comprising: reacting a hydroxy-substituted aromatic compound with a base comprising a metal cation in an aqueous medium to provide a mixture comprising water and a metal salt of the hydroxy-substituted aromatic compound; contacting the mixture with a substantially water-immiscible solvent at a temperature greater than the boiling point of water at a prevailing pressure; partially removing water and the water-immiscible solvent from the contacted mixture to provide a water-immiscible, solvent-rich phase comprising the metal salt of the hydroxy-substituted aromatic compound and the water-immiscible solvent; introducing an optionally substituted C₁₋₆ aliphatic alcohol to the water-immiscible phase to provide a solution; and separating the water and the isopropanol from the solution to provide a slurry comprising the metal salt of the hydroxy-substituted aromatic compound and the water-immiscible solvent.

Embodiment 3: A method for the manufacture of a metal salt of a hydroxy-substituted aromatic compound, the method comprising: reacting a hydroxy-substituted aromatic compound with a base comprising a metal cation in an organic medium comprising isopropanol, to provide a mixture comprising a metal salt of the hydroxy-substituted aromatic compound, an optionally substituted C₁₋₆ alcohol, and water produced from the reaction between the hydroxy-substituted aromatic compound and the base; contacting the mixture with a substantially water-immiscible solvent at a temperature greater than the boiling point of water at a prevailing pressure to provide a mixture further comprising the substantially water-immiscible solvent; and removing water and the alcohol from the mixture further comprising the substantially water-immiscible solvent, to provide a slurry comprising the metal salt of the hydroxy-substituted aromatic compound and the water-immiscible solvent.

Embodiment 4: The method of any one of Embodiments 1 to 3, wherein the slurry comprises less than 500, less than 250, less than 100, less than 50, preferably less than 20 ppm of water.

Embodiment 5: The method of any one or more of Embodiments 1 to 4, wherein the method further comprises: introducing a polar aprotic solvent to the slurry, wherein the polar aprotic solvent has a boiling point greater than the boiling point of the water-immiscible solvent; and removing the water-immiscible solvent from the slurry containing the polar aprotic solvent, to provide a composition comprising the polar aprotic solvent and the metal salt of the hydroxy-substituted aromatic compound.

Embodiment 6: The method of Embodiment 5, wherein the polar aprotic solvent is diphenyl sulfone, sulfolane, dimethyl sulfoxide, N-methyl-2-pyrrolidone, dimethyl formamide, DMAc, or a combination comprising at least one of the foregoing.

Embodiment 7: The method of any one or more of Embodiments 1 to 6, wherein the aliphatic alcohol is isopropanol.

Embodiment 8: The method of any one or more of Embodiments 1 to 7, wherein the hydroxyl-substituted aromatic compound is of the formula

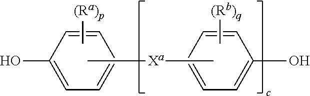

wherein R^{a} and R^{b} are each independently a halogen atom or a monovalent C₁₋₁₂ hydrocarbon group, p and q are each independently integers of 0 to 4, c is zero to 4, and X^{a} is a single bond, —O—, —S—, —S(O)—, —S(O)₂—, —C(O)—, or a C₁-₁₈ organic bridging group; and the base is an alkali metal hydroxide, an alkaline carbonate, an alkali bicarbonate, or a combination comprising at least one of the foregoing.

Embodiment 9: The method of any one or more of Embodiments 1 to 8, wherein the hydroxyl-substituted aromatic compound is 2,2-bis(4-hydroxyphenyl)propane or 4,4′-dihydroxybiphenyl; and the base is sodium hydroxide or potassium hydroxide.

Embodiment 10: The method of any one or more of Embodiments 1 to 9, wherein the water-immiscible solvent comprises benzene, toluene, xylene, phenetole, anisole, veratrole, diphenylsulfone, chlorobenzene, bromobenzene, ortho-dichlorobenzene, 1,3-dichlorobenzene, 1,4-dichlorobenzene, 1,3,5-trichlorobenzene, 1,2,4-trichlorobenzene, or a combination comprising at least one of the foregoing.

Embodiment 11: The method of any one or more of Embodiments 1 to 10, wherein the water-immiscible solvent comprises ortho-dichlorobenzene.

Embodiment 12: A method for the manufacture of a polyetherimide composition, the method comprising polymerizing a bis(N-(substituted phthalimido))aromatic compound and an alkali metal salt of a dihydroxy aromatic compound of any one of Embodiments 1 to 10 to form a polyetherimide composition.

Embodiment 13: The method of Embodiment 12, wherein the polymerizing is carried out without a phase transfer catalyst.

Embodiment 14: The method of Embodiment 12, wherein the polymerization is carried out in the presence of a phase transfer catalyst.

The singular forms “a,” “an,” and “the” include plural referents unless the context clearly dictates otherwise. “Or” means “and/or.” The endpoints of all ranges directed to the same component or property are inclusive and independently combinable. Unless defined otherwise, technical and scientific terms used herein have the same meaning as is commonly understood by one of skill in the art to which this invention belongs. As used herein, a “combination” is inclusive of blends, mixtures, alloys, reaction products, and the like.

Compounds are described using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through carbon of the carbonyl group.

As used herein, the term “hydrocarbyl” includes groups containing carbon, hydrogen, and optionally one or more heteroatoms (e.g., 1, 2, 3, or 4 atoms such as halogen, O, N, S, P, or Si). “Alkyl” means a branched or straight chain, saturated, monovalent hydrocarbon group, e.g., methyl, ethyl, i-propyl, and n-butyl. “Alkylene” means a straight or branched chain, saturated, divalent hydrocarbon group (e.g., methylene (—CH₂—) or propylene (—(CH₂)₃—)). “Alkenyl” and “alkenylene” mean a monovalent or divalent, respectively, straight or branched chain hydrocarbon group having at least one carbon-carbon double bond (e.g., ethenyl (—HC═CH₂) or propenylene (—HC(CH₃)═CH₂—). “Alkynyl” means a straight or branched chain, monovalent hydrocarbon group having at least one carbon-carbon triple bond (e.g., ethynyl). “Alkoxy” means an alkyl group linked via an oxygen (i.e., alky-O—), for example methoxy, ethoxy, and sec-butyloxy. “Cycloalkyl” and “cycloalkylene” mean a monovalent and divalent cyclic hydrocarbon group, respectively, of the formula —CₙH₂ₙ₋ₓ and —CₙH₂ₙ₋₂ₓ— wherein x is the number of cyclization(s). “Aryl” means a monovalent, monocyclic or polycyclic aromatic group (e.g., phenyl or naphthyl). “Arylene” means a divalent, monocyclic or polycyclic aromatic group (e.g., phenylene or naphthylene). The prefix “halo” means a group or compound including one more halogen (F, Cl, Br, or I) substituents, which can be the same or different. The prefix “hetero” means a group or compound that includes at least one ring member that is a heteroatom (e.g., 1, 2, or 3 heteroatoms, wherein each heteroatom is independently N, O, S, or P.

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents and/or variables are permissible provided that the substitutions do not significantly adversely affect synthesis or use of the compound. Groups that can be present on a substituted position include (—NO₂), cyano (—CN), hydroxy (—OH), halogen, thiol (—SH), thiocyano (—SCN), C₂-₆ alkanoyl (e.g., acyl (H₃CC(═O)—); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxy; C₆₋₁₀ aryloxy such as phenoxy; C₁₋₆ alkylthio; C₁₋₆ or C₁₋₃ alkylsulfinyl; C1-6 or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 11 to 3 separate or fused rings and from 6 to 18 ring carbon atoms.

All references cited herein are incorporated by reference in their entirety. While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A method for the manufacture of a metal salt of a hydroxy-substituted aromatic compound, the method comprising:
reacting a hydroxy-substituted aromatic compound with a base comprising a metal cation in an aqueous medium to provide a mixture comprising water and a metal salt of the hydroxy-substituted aromatic compound;
contacting the mixture with a substantially water-immiscible solvent at a temperature greater than the boiling point of water at a prevailing pressure;
introducing an optionally substituted C₁₋₆ aliphatic alcohol; and
removing water and the alcohol to provide a slurry comprising the metal salt of the hydroxy-substituted aromatic compound and the water-immiscible solvent.

2. A method for the manufacture of a metal salt of a hydroxy-substituted aromatic compound, the method comprising:
reacting a hydroxy-substituted aromatic compound with a base comprising a metal cation in an aqueous medium to provide a mixture comprising water and a metal salt of the hydroxy-substituted aromatic compound;
contacting the mixture with a substantially water-immiscible solvent at a temperature greater than the boiling point of water at a prevailing pressure;
partially removing water and the water-immiscible solvent from the contacted mixture to provide a water-immiscible, solvent-rich phase comprising the metal salt of the hydroxy-substituted aromatic compound and the water-immiscible solvent;
introducing an optionally substituted C₁₋₆ aliphatic alcohol to the water-immiscible phase to provide a solution; and
separating the water and the isopropanol from the solution to provide a slurry comprising the metal salt of the hydroxy-substituted aromatic compound and the water-immiscible solvent.

3. A method for the manufacture of a metal salt of a hydroxy-substituted aromatic compound, the method comprising:
reacting a hydroxy-substituted aromatic compound with a base comprising a metal cation in an organic medium comprising isopropanol, to provide a mixture comprising a metal salt of the hydroxy-substituted aromatic compound, an optionally substituted C₁₋₆ alcohol, and water produced from the reaction between the hydroxy-substituted aromatic compound and the base;
contacting the mixture with a substantially water-immiscible solvent at a temperature greater than the boiling point of water at a prevailing pressure to provide a mixture further comprising the substantially water-immiscible solvent; and
removing water and the alcohol from the mixture further comprising the substantially water-immiscible solvent, to provide a slurry comprising the metal salt of the hydroxy-substituted aromatic compound and the water-immiscible solvent.

4. The method of claim 1, wherein the slurry comprises less than 500 ppm of water.

5. The method of claim 1, wherein the method further comprises:
introducing a polar aprotic solvent to the slurry, wherein the polar aprotic solvent has a boiling point greater than the boiling point of the water-immiscible solvent; and
removing the water-immiscible solvent from the slurry containing the polar aprotic solvent, to provide a composition comprising the polar aprotic solvent and the metal salt of the hydroxy-substituted aromatic compound.

6. The method of claim 5, wherein the polar aprotic solvent is diphenyl sulfone, sulfolane, dimethyl sulfoxide, N-methyl-2-pyrrolidone, dimethyl formamide, DMAc, or a combination comprising at least one of the foregoing.

7. The method of claim 1, wherein the aliphatic alcohol is isopropanol.

8. The method of claim 1, wherein the hydroxyl-substituted aromatic compound is of the formula 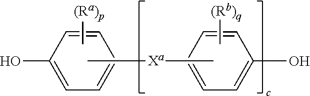
wherein R^{a} and R^{b} are each independently a halogen atom or a monovalent C₁₋₁₂ hydrocarbon group, p and q are each independently integers of 0 to 4, c is zero to 4, and X^{a} is a single bond, —O—, —S—, —S(O)—, —S(O)₂—, —C(O)—, or a C₁₋₁₈ organic bridging group; and
the base is an alkali metal hydroxide, an alkaline carbonate, an alkali bicarbonate, or a combination comprising at least one of the foregoing.

9. The method of claim 1, wherein
the hydroxyl-substituted aromatic compound is 2,2-bis(4-hydroxyphenyl)propane or 4,4′-dihydroxybiphenyl; and
the base is sodium hydroxide or potassium hydroxide.

10. The method of claim 1, wherein the water-immiscible solvent comprises benzene, toluene, xylene, phenetole, anisole, veratrole, diphenylsulfone, chlorobenzene, bromobenzene, ortho-dichlorobenzene, 1,3-dichlorobenzene, 1,4-dichlorobenzene, 1,3,5-trichlorobenzene, 1,2,4-trichlorobenzene, or a combination comprising at least one of the foregoing.

11. The method of claim 1, wherein the water-immiscible solvent comprises ortho-dichlorobenzene.

12. A method for the manufacture of a polyetherimide composition, the method comprising
polymerizing a bis(N-(substituted phthalimido))aromatic compound and an alkali metal salt of a dihydroxy aromatic compound of claim 1 to form a polyetherimide composition.

13. The method of claim 12, wherein the polymerizing is carried out without a phase transfer catalyst.

14. The method of claim 12, wherein the polymerization is carried out in the presence of a phase transfer catalyst.

15. The method of claim 2, wherein the method further comprises:
introducing a polar aprotic solvent to the slurry, wherein the polar aprotic solvent has a boiling point greater than the boiling point of the water-immiscible solvent; and
removing the water-immiscible solvent from the slurry containing the polar aprotic solvent, to provide a composition comprising the polar aprotic solvent and the metal salt of the hydroxy-substituted aromatic compound.

16. The method of claim 2, wherein the polar aprotic solvent is diphenyl sulfone, sulfolane, dimethyl sulfoxide, N-methyl-2-pyrrolidone, dimethyl formamide, DMAc, or a combination comprising at least one of the foregoing.

17. The method of claim 2, wherein the aliphatic alcohol is isopropanol.

18. The method of claim 3, wherein the method further comprises:
introducing a polar aprotic solvent to the slurry, wherein the polar aprotic solvent has a boiling point greater than the boiling point of the water-immiscible solvent; and
removing the water-immiscible solvent from the slurry containing the polar aprotic solvent, to provide a composition comprising the polar aprotic solvent and the metal salt of the hydroxy-substituted aromatic compound.

19. The method of claim 3, wherein the polar aprotic solvent is diphenyl sulfone, sulfolane, dimethyl sulfoxide, N-methyl-2-pyrrolidone, dimethyl formamide, DMAc, or a combination comprising at least one of the foregoing.

20. The method of claim 3, wherein the aliphatic alcohol is isopropanol.
